(19) United States
(12) Patent Application Publication
(10) Pub. No.: US 2014/0121687 A1
(43) Pub. Date: May 1, 2014

(54) HORIZONTAL PHLEBECTOMY DEVICE

(71) Applicant: VenX LLC, Nashville, TN (US)

(72) Inventors: Chris Genau, Seattle, WA (US); Edward M. Boyle, Bend, OR (US); Andrew Jones, Bend, OR (US)

(73) Assignee: VenX LLC, Nashville, TN (US)

(21) Appl. No.: 13/935,642

(22) Filed: Jul. 5, 2013

Related U.S. Application Data
(60) Provisional application No. 61/668,138, filed on Jul. 5, 2012.

(51) Int. Cl. A61B 17/00

(52) U.S. Cl. CPC A61B 17/00008
USPC 606/159

(57) ABSTRACT

A surgical tool for the obliteration of small and medium sized veins in the skin of a patient, through the deployment of a hook member.

### CROSS REFERENCE

The present case claims the benefit of and is the utility conversion of U.S. Provisional Application 61/668,138 filed Jul. 5, 2012, entitled “Horizontal Phlebectomy—A Novel Device for the Treatment of Small and Medium Sized Veins”.

### FIELD OF THE INVENTION

The present invention relates generally to vein obliteration technology and more particularly to a surgical instrument operated by a physician for locating, intercepting and obliterating small and medium sized veins in the skin of a patient.

### BACKGROUND OF THE INVENTION

The heart pumps blood to supply oxygen and nutrients to all parts of the body. Arteries carry blood from the heart towards the body parts, while veins carry blood from the body parts back to the heart. Veins contain one-way valves to prevent the blood from flowing backwards. If the one-way valve becomes weak, some of the blood can leak backwards through the valve, collect in the vein upstream of the valve, and then become congested as the pressure builds. This congestion will cause the vein to abnormally enlarge. These enlarged veins can be seen on the surface of the skin. Removal of diseased veins can be accomplished in several ways including surgical intervention.

### SUMMARY OF THE INVENTION

The present invention relates generally to a tool that can be used conveniently by a physician or other user to intercept and pull a vein to disrupt the vessel. In contrast to prior art devices that simply cut the vein; the present device includes a hook that engages the vein with blunt, non-cutting surfaces. Motion of the tool supplies force to the vein and the tissues surrounding the vein. The tool is manipulated enough to disrupt the vein and create an extensive injury to the vein and the immediate tissue at the site of the intervention. This form of injury prevents the vein from re-cannulizing and therefore results in vein obliteration. The applicants have discovered that this distributed injury results in the more reliable removal of the vein than the previously known vein cutting technique. It is preferred that the tool enter the skin nearly “horizontally” to facilitate vein location and obliteration. The exact angle is a matter of medical judgment by the tool in tended to operate at low entry angles.

### BRIEF DESCRIPTION OF THE DRAWINGS

Throughout the figures of the drawing identical reference numerals indicate identical structure, wherein:

FIG. 1 shows the device in the hand of a physician user interacting with a patient;

FIG. 2 shows the interior of the device;

FIG. 3A shows a portion of the device in isolation; and,

FIG. 3B shows a portion of the device in isolation.

### DETAILED DESCRIPTION OF THE INVENTION

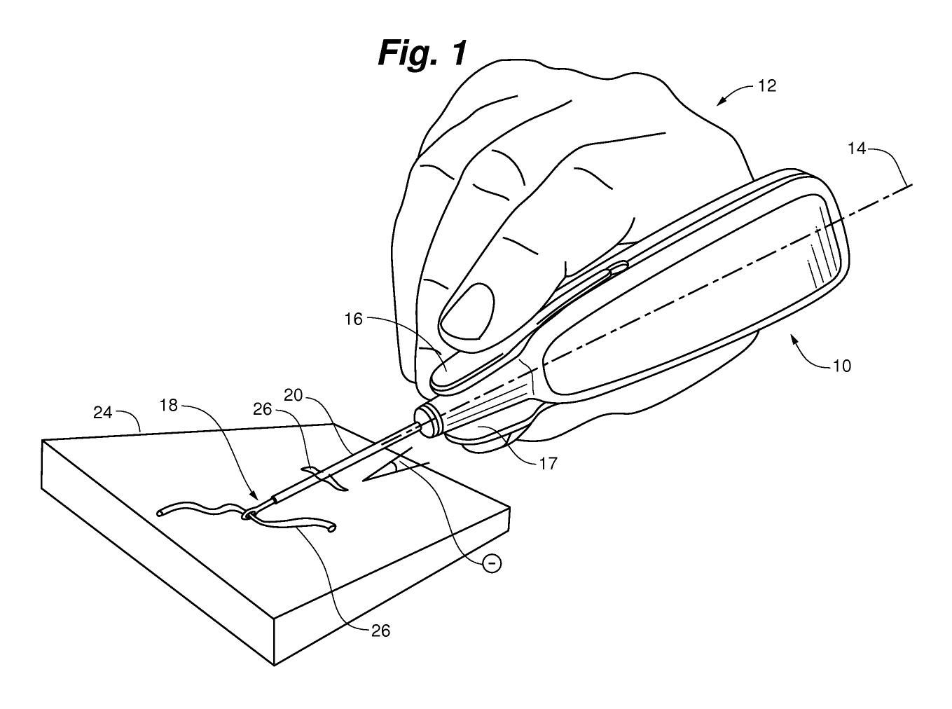

FIG. 1 shows the device 10 resting in the hands of a physician 12. The device lies in the hand between the thumb and forefinger. The device has an elongate axis 14 and a set of finger paddle levers typified by lever 16 and lever 17 for activation by the physician's fingers. The physician squeezes the two lever arms together to activate the device. The energy supplied by translating the motion of the physician's fingers causes a hook member 18 to emerge from a needle member 20.

In use the needle member 20 is forced into the patients skin 24 through a wound 26. Activation of the hook member 18 and manipulation by the physician 12 allows the hook member 18 to engage and snare a vein 26 located below the skin 24 and tear it by manipulation of the lever 16 and lever 17. Entry into the skin occurs at a low angle indicated as theta in the figure. Full compression of the finger levers moves the device from a static initial state or position to a competed or deployed position locating the hook member 18 in its fully extended a fixed position a predetermined distance into the tissue.

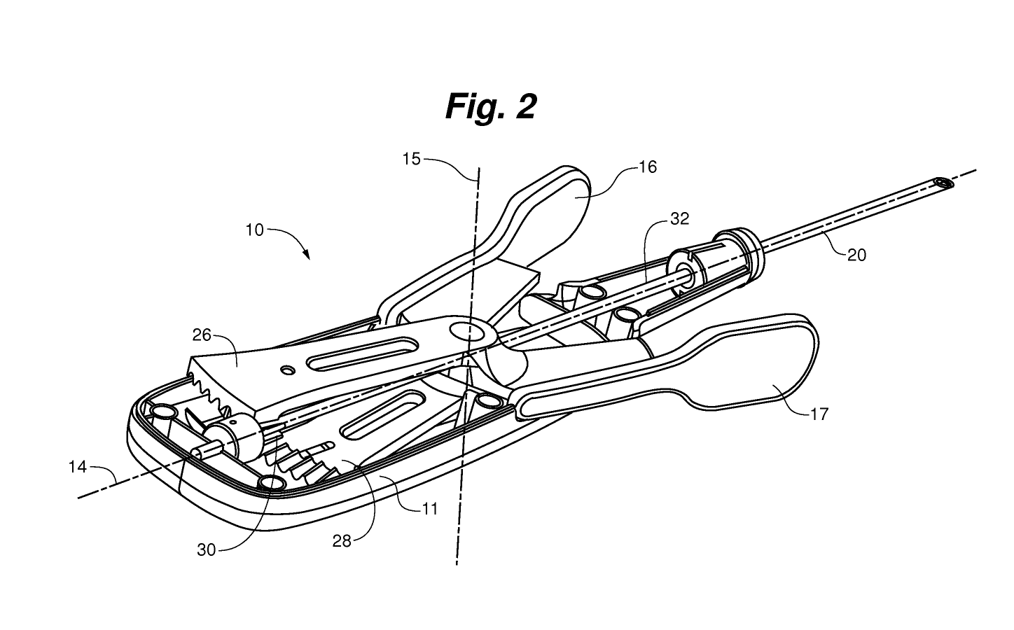

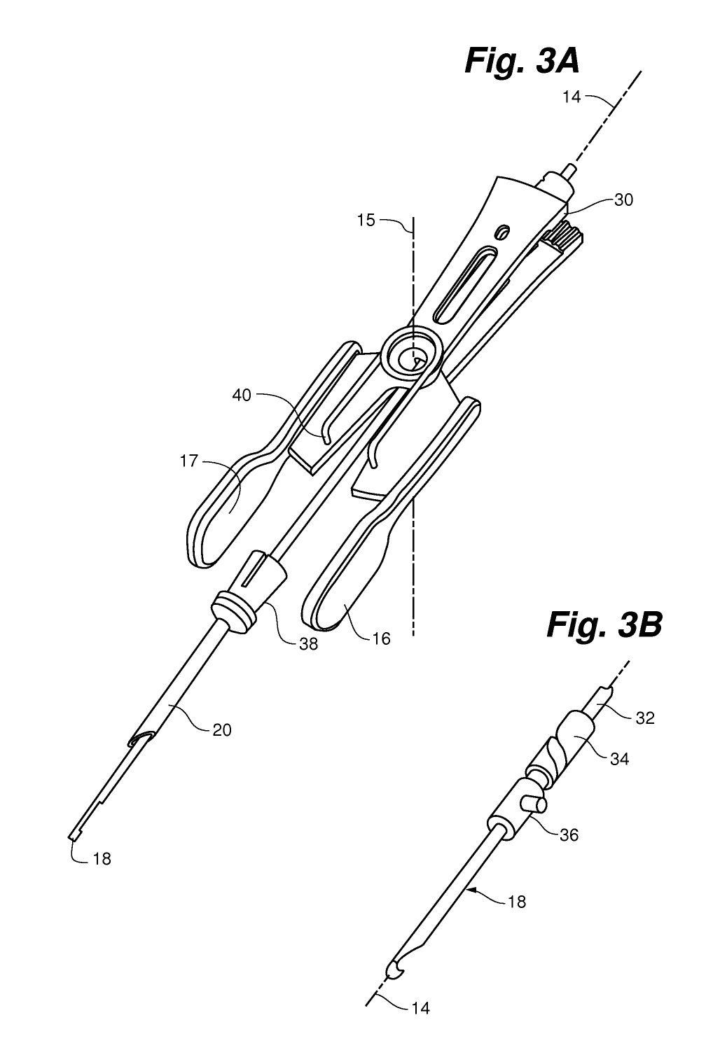

FIG. 2 shows a perspective view of the interior of the device 10 and shows one embodiment of a mechanical arrangement for translating the surgeon's squeezing of the paddle lever arms, rotating them about axis 14 to a translational movement of the hook member 18. Each finger paddle lever 16 and 17 connects to a sector gear contained within the housing bottom. The sector gear for lever 17 is labeled 26 in the figure. The complimentary sector gear 28 is formed integrally with lever arm 16. Both finger lever arms share a common axis of rotation 15 or common axle. Both arms are mounted for pivoting around this common axle that is perpendicular to axis 14. The finger lever arms and associated sector gear segments are biased against each other by a wound torsion spring 40, shown in FIG. 3B. The housing or body shown is half of a clamshell construction the forms the body 11 of the device 10.

FIG. 3A shows a portion of the mechanism in isolation to improve clarity of description of the operation. As seen in FIG. 3A the spring 40 is anchored in both lever arm 16 and 17 at locations defined by holes. This arrangement causes the lever arms to react against each other to improve the “feel” or sensitivity of the device. It is believed that the haptics of the device will be important to its acceptance and use. As the two gear sectors scissor past each they drive a pinion gear 30 rotating about axis 14. This pinion gear is coupled to shaft 32 that connects to the tip assembly bushing 38. The pinion gear 30 and shaft 32 are journaled in bearings not labeled that serve to retain the shaft within the housing or body while still allowing smooth rotation. The pinion gear further serves to keep the levers mechanically mated to each other in synchronous fashion such that pivoting or actuating movement of one lever is dependent on movement of the second lever. In summary, lever motion results in rotation of the shaft 32 and a restoring force supplied by spring 40 returns the levers to the initial position.

The sector gear tooth count and pitch to pinion diameter pitch and tooth count establish a ratio that is selected to provide the maximum desired amount of rotation that is translated into a translational motion of the hook member 18.

FIG. 3B is a view of a portion of the device 10 illustrating the mechanism to advance the hook member 18. A worm and peg drive system is provided to rotate the worm section 34 when the shaft 32 rotates. A peg 36 located in the groove of the worm 34 advances or retracts based on this motion. The peg 36 also sits in an anti-rotation groove in bushing 38 to prevent rotation of the hook member 18 as it advances out of the needle member 20.

In summary the device 10 has an instrument body 11 that is held in a physician's hand 12. His fingers may operate a pair of opposed levers 16 and 17.

When pressed the levers rotate a spindle assembly that advance a hook member 18 out of the needle member 20. As a part of the treatment the device 10 handle may be retracted and or rotated with the hook 18 engaging a vein 26 as shown in FIG. 1.

To permit entry into the skin a needle member 20 will be quite sharp. In use the hypodermic needle member 20 will cover the hook member 18 during entry into the skin 24. The hook member 18 will emerge from the needle member 20 and surround or hook the vein 26. Rotation of the handle and retraction of the hook will disrupt the vein 26.

The device of the present invention has two mechanisms of action. A translational mode and a rotational mode. They may be used together or separately to effectively remove a vein.

In general the device includes a handle assembly that can rotate a spindle to advance a hook.

The physician can also pull on the spindle or push on the spindle with the handle. The spindle can retract into a hypodermic needle that can be manipulated by the handle.

In use the physician enters the skin at a shallow angle with the spindle retracted into the handle thereby exposing the sharp needle tip. The needle is manipulated to navigate to the vein and the spindle extended to engage the vein.

With the vein trapped in the hook of the distal tip of the spindle the physician may apply both traction and/or rotation to ensure that a long length of vein is injured.

Important attributes of the device include the convertibility between sharp and blunt dissection along with two modes of vein disruption.

It is also anticipated that fluid injection through the needle or the spindle to provide local anesthetic effects may be readily incorporated into the device.

The device is intended to be a single use tool however reusable versions are contemplated within the scope of the invention. It is proposed to have single tip configuration but a replaceable tip is contemplated within the scope of the invention.

What is claimed is:

1. A surgical tool for obliterating a vein comprising:
a stationary body adapted to be held and manipulated by a hand;
a first lever arm adapted to be actuated by a finger;
a second lever arm adapted to be actuated by a finger;
said first and second lever arms are opposed on the sides of said body;
an axle passing though said first and second lever arms forming a pivot for each lever arm and anchoring the lever arms in said body;
a first gear sector coupled to said first lever arm;
a second gear sector coupled to said second lever arm;
a pinion gear meshed with each gear sector to translate motion of the lever arms into a rotational motion of a pinion shaft;
a worm screw coupled to said pinion shaft;
a peg attached to a hook member;
said worm screw and peg engaging together to translate rotational motion of said pinion shaft into longitudinal motion of said hook member;
said tip assembly having a hook member with blunt surfaces adapted to straddle and capture a vein;
whereby lever arm motion about the pivot extends the hook member permitting the obliterating of a vein by mechanical trauma.

2. The tool of claim 1 further including a spring connecting said first and second lever arms to provide a restoring force to return said lever arms to an initial position.

3. The tool of claim 1 wherein said gear sector and pinion gear together limit to the maximum extension of said hook member.

4. The tool of claim 1 further including a needle member attached to said handle and having a lumen for enshrouding said hook member.
